(19) United States
(12) Patent Application Publication
(10) Pub. No.: US 2025/0045690 A1
(43) Pub. Date: Feb. 6, 2025

(54) SYSTEMS AND DEVICES TO MONITOR PERISHABLE PRODUCTS

(71) Applicants: Asusena Huezo, Los Angeles, CA (US); Fadi J. Sayegh, Los Angeles, CA (US); Mark A. Chatoff, Los Angeles, CA (US); Shanon L. Thomas, Los Angeles, CA (US)

(72) Inventors: Asusena Huezo, Los Angeles, CA (US); Fadi J. Sayegh, Los Angeles, CA (US); Mark A. Chatoff, Los Angeles, CA (US); Shanon L. Thomas, Los Angeles, CA (US)

(21) Appl. No.: 18/229,591

(22) Filed: Aug. 2, 2023

(51) Int. Cl. G06Q 10/087; G01N 33/02

(52) U.S. Cl. CPC G06Q 10/087; G01N 33/02

(57) ABSTRACT

Devices and systems to monitor, analyze, and report the state and condition of perishable products, are disclosed. In a core embodiment, a device includes a housing incorporating a data display, a data communication receiver and transmitter, a data processor operating software, and having data communication connections with the data display, data receiver and transmitter, and at least one remote user display device. The device monitors and is capable of reporting metrics relating to the perishable products. In other embodiments, the device monitors and analyzes certain metrics of the perishable products, and reports such analyzed metrics and conditions to a remote user display device. In a further system embodiment, the system includes a plurality of sensors to monitor and record one or more metrics relating to perishable products, including expiration date, weight, temperature, and humidity, with such metrics being used to analyze the state and condition of the products.

### FIELD OF THE INVENTION

The present invention generally relates to the field of systems, devices, and methodologies used to monitor perishable products, including perishable food products and consumable liquids. More particularly, the present invention relates to, at its core, a device that is capable of data communication with remote user devices, and having a display to show relevant status or metrics associated with the perishable food products that are being monitored or managed by the device. Further embodiments of the present invention, are systems and devices incorporating a plurality of sensors used to sense a plurality of metrics associated with the status and condition of perishable food products, with such systems and devices further incorporating compute processing capability, and the associated execution of analytics relating to the food products, as well as communication capability for transmitting sensed data and/or some analytics to the cloud, for further subsequent cloud compute analytics. Such systems and devices can provide detailed analysis of the plurality of metrics, and then record and report the status and condition of the perishable food products that are monitored. In certain embodiments, the metrics sensed and analyzed include various expiration dates, “use-by” dates, “best-by” dates, weight, volume, color, and local gases emitted by the perishable food products.

### BACKGROUND DESCRIPTION AND REVIEW OF CERTAIN PRIOR ART

Food spoilage has been, and continues to be, a vexing problem. While refrigerators, freezers, and various packaging innovations have provided substantial advancements in extending the shelf-life of many perishable food products, consumers still throw away a substantial amount of food, some of which is has not exceeded its shelf-life, and may well still be safely consumable.

Some primary reasons that stored food products reach their shelf-life, and are, or are not disposed of, are that (a) users of such food products are not aware that such food is reaching the end of its shelf-life, and/or (b) users do not know whether the food items have in fact reached a true, accurate, and safe shelf-life. With respect to the first reason, many food items are lost in the mix of what is stored in a refrigerator or in a cupboard. With respect to the second reason, consumers are either not provided with an accurate measure of food item shelf-life, and/or consumers tend to be safety conscience, and throw away food items that the consumer is not sure have reached the end of their shelf-life.

What is needed is a means to provide consumers with an accurate means to monitor the condition of perishable food items that are being stored. While various devices and systems have been created to address this issue and provide consumers with the status of certain perishable food items, such devices and systems have not been widely accepted because such devices do not fully address the noted problems, and do not provide a cost effective and readily implementable solution.

While others have designed different devices, apparatus, and system to monitor the condition of perishable food products, some of which may appear to address some of the issues and problems identified above, none of these designs fully address or resolve the noted problems.

More particularly, U.S. Pat. No. 9,712,893 for a Sensing Device and Method To Monitor Perishable Goods, by Warkentin et al., teaches a sensing device that monitors conditions in a local environment and wirelessly communicates those conditions. The sensing device has a condition sensor, a processor, a memory and a communication device, a motion sensor and an energy source. The '893 patent does not disclosure or teach an ability to record and monitor expiration or use-by dates, nor does the '893 patent disclose any form of inventory monitoring or control.

Further, U.S. patent application Ser. No. 12/701,151 for a Refrigerated Container Monitoring System, filed by McIlvain et al., teaches a monitoring system for a refrigerated container carrying temperature-sensitive cargo. The system includes sensors disposed throughout the container and/or an associated vehicle for measuring parameters of the container or vehicle. While the '151 application does include sensors to monitor and measure certain parameters relating to refrigerated and temperature-sensitive cargo, the system does not disclose or teach the ability to monitor expiration dates, or product condition or status, or providing any means of inventory control through product monitoring.

Similarly, U.S. Pat. No. 7,784,707 for an Environmental Condition Monitoring Of A Container, invented by Witty et al., and owned by Xata Corporation, relates to trucking or logistics monitoring systems. The '707 patent teaches a device that includes an environmental condition sensor, a light sensor, and a control module, where the control module associates a light intensity above a defined threshold with an open-door status of a container. As designed, the Witty et al. system provides techniques for monitoring environmental conditions within a container, such as a refrigerated trailer. However, the '707 patent does not disclose or teach any element of monitoring expiration dates, or product condition or status, or inventory monitoring.

### SUMMARY OF THE INVENTION

The present invention overcomes the disadvantages of the prior art and fulfills the needs of users as described above by providing monitoring and reporting devices and systems, for monitoring and reporting the status and condition of perishable goods, including perishable food items.

One preferred embodiment of the invention is a device for reporting status of perishable items, said device comprising, a housing with each of the following incorporated into said housing, a data communications receiver, a data communication transmitter; and a data processor operating machine-readable media in data communication with said data communications receiver, and said data communications transmitter, and a power source for said data communications receiver, data communications transmitter, and said data processor; wherein said device is in data communication with at least one remote user display device.

A second embodiment of the invention is a device for reporting status of perishable items, said device comprising, a housing with each of the following incorporated into said housing, a data communications receiver, a data communication transmitter, a data display, and a data processor operating machine-readable media in data communication with said data communications receiver, and said data communications transmitter; and a power source for said data communications receiver, data communications transmitter, data display, and said data processor; wherein said device is in data communication with at least one remote user display device.

A third embodiment of the invention is a device to monitor, analyze, record, and report condition and status of perishable food items, said device, comprising, a housing, a data input device for manually entering metric data associated with said food items into said cap/display, a data display device, a data communications transceiver, a processor operating machine-readable media executing at least one algorithm for analyzing metrics associated with said food items, a power source for said data display, data communications transceiver, and said data processor, and wherein said device is in data communication with at least one remote user display device.

A fourth embodiment of the invention is a device to monitor, analyze, and report condition and status of perishable food items, said device, comprising, an enclosure having a sealable opening, said enclosure defining an internal volume, a plurality of sensors located within said sealable top to sense and monitor metrics associated with said condition and status of said perishable food items, wherein said plurality of sensors sense at least one of a group of metrics associated with said perishable food items, said at least one of a group of metrics comprising expiration date, weight, volume, color of the perishable food items, and temperature, humidity, and emitted gases within said enclosure internal volume, such as methane, oxygen, and/or carbon dioxide, a microprocessor in data communication with each of said plurality of sensors, wherein said microprocessor records and analyzes a condition and status of said perishable food items based upon at least one of said sensed metrics, a transmitter in data communication with said microprocessor, a power source for said plurality of sensors, microprocessor and transmitter; and wherein said device is in data communication with at least one remote user display device and receiver, wherein said device transmitter transmits said condition and status of said perishable food items to said at least one remote user display device and receiver.

### BRIEF DESCRIPTION OF THE DRAWINGS OF CERTAIN EMBODIMENTS

For the purposes of illustrating the invention, the attached drawings show certain aspects and embodiments that are presently preferred. However, it should be understood that the invention is not limited to the precise configurations or system elements, or process steps as shown in the accompanying drawings, but rather is further disclosed and claimed according to the attached claims.

FIG. 1 is a system diagram of an embodiment of the monitoring device configured as a housing cap with a receiver/transmitter and processor, all in communication with remote users;

FIG. 2 is a system diagram of an embodiment of the monitoring device configured as a housing cap with a receiver/transmitter, a processor, and a display, all in communication with remote users;

FIG. 3 is a system diagram of an embodiment of the monitoring device configured as a housing cap with a receiver/transmitter and processor, a display, and incorporating at least one sensor, all in communication with remote users;

FIG. 4 is a system diagram of an embodiment of the monitoring device configured as a housing cap with a combined transceiver, processor, display, and incorporating at least one sensor, all in communication with remote users;

FIG. 5 is an illustration of an embodiment of the monitoring and reporting device configured as a sealable container;

FIG. 6 is an illustration of an embodiment of the monitoring and reporting device configured as a plate or scale;

FIG. 7 is an illustration of an embodiment of the monitoring and reporting device incorporating an evacuation element and bacterial control lighting; and

FIG. 8 is an illustration of an embodiment of the monitoring and reporting device incorporating a motion detector and a lighting element.

### DETAILED DESCRIPTION OF CERTAIN EMBODIMENTS

Devices and systems that are used to monitor, analyze, record, and report the state and condition of various perishable products, including food products and consumable liquids, are described and disclosed.

In different core exemplary embodiments, the device may comprise a housing incorporating a data communication receiver and transmitter (or transceiver), a data processor that is operating pre-programmed software, with the data processor having data communication connections with the data receiver and transmitter (or transceiver), and with the data receiver and data transmitter (or transceiver) having a communications connection to at least one remote user display or interface device. In a further embodiment, the device may further comprise and include a data display in the device housing to provide users with direct information or data presentation capability separate from a remote user display or interface device.

In certain exemplary embodiments, the monitoring devices are capable of reporting certain relevant metrics relating to the perishable food products. As described in this specification, the metrics may be sensed or measured, or such metrics may be directly calculated based upon known or previously entered data.

At a basic level, the monitoring devices may simply record and report expiration or “best by” dates. In other more advanced embodiments, the monitoring devices may monitor and analyze various metrics associated with the perishable products that are being monitored, and then report such analyzed metrics and conditions to at least one remote user display device.

In a further system embodiment, a system may include a plurality of sensors incorporated into one or more monitoring devices, to monitor and record one or more metrics relating to the perishable products. By way of example, such metrics may include one or more of product expiration date, “use-by” date, weight, volume, temperature, humidity, and/or surrounding air/gas composition, with some or all of such metrics being used to analyze the state and condition of the perishable products.

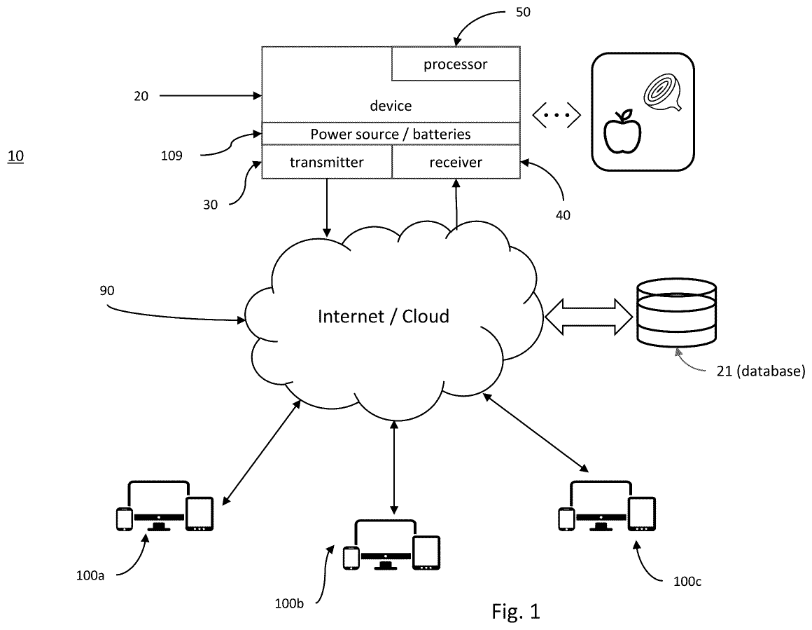

At its core, the monitoring device 10 monitors relevant data associated with products or goods, including perishable food items, and to provide such data to one or more users having data communication connections with the monitoring device 10. Accordingly, in a core embodiment, as illustrated in FIG. 1, shown in a block diagram/parametric format, the monitoring device 10 comprises as primary elements a housing 20 with a data transmitter 30, a data receiver 40, and a data or micro-processor 50, with each element incorporated into the housing 20. The data transmitter 30 and data receiver 40 are further wirelessly connected to and communicating with a global communications network, or the Internet 90, such that data from the monitoring device 10 may be transmitted to, and/or received from at least one remote user display device 100a, 100b, 100c. By way of one example, such remote user display devices 100a, 100b, 100c may be a smartphone or a tablet.

The type of data that may be transmitted from and to the monitoring device 10 ranges from simple counters or timers, all the way to more complicated, sensor dependent analytics such as product weight, volume, color, shape, and internally sensed gases.

On the basic or core end of the spectrum, the data provided to the monitoring device could be expiration dates, or “use-by” dates, which may be entered by a user through his or her user device 100a, which is then entered into the data processor 50. Once entered into the data processor 50, a countdown timer or clock, within the data processor 50, may commence a countdown or timeline, and provide updates of the countdown to the user through the users' remote display device 100a. The data processor 50 may also execute algorithms that output trigger alerts or messages to the data transmitter 30, that are then transmitted to connected user display devices 100a, 100b, 100c, to alert one or more users of the occurrence of trigger events, such as expiration dates, or “use-by” dates that have passed or have been exceeded. The algorithms may also provide triggers for, and transmit messages of interim dates passing, such as “one week remaining for usage” or “one-day remaining for product life.”

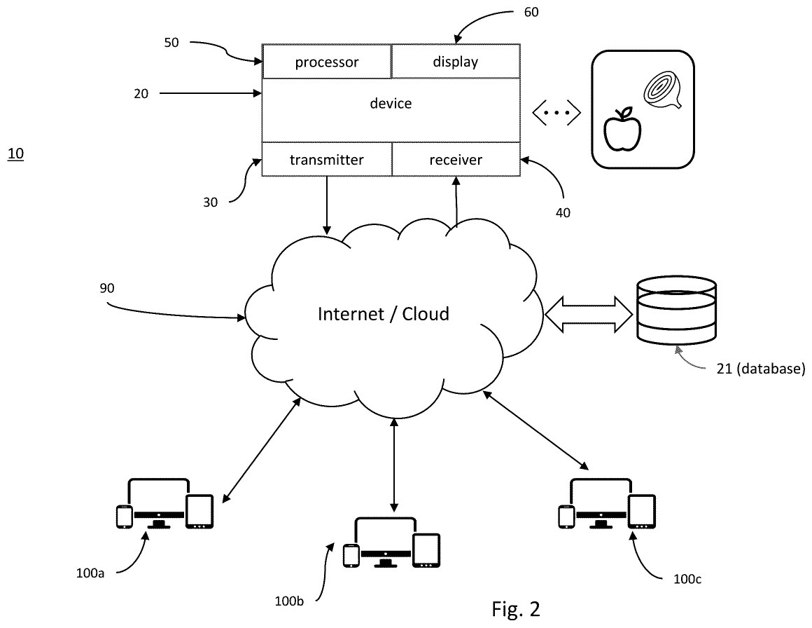

A further embodiment, illustrated in FIG. 2, again in a block diagram format, has a monitoring device 10 that comprises a housing 20 with an incorporated data transmitter 30, data receiver 40, a data processor 50, and a data display 60. The data display 60 may be a low-energy, liquid crystal display (“LCD”), that provides the user with relevant monitoring device output information. Alternatively, the data display 60 may be a more capable user interface, such as a touch screen similar to a smartphone or tablet-type device.

The monitoring device 10 also incorporates a power source 109, as illustrated in FIG. 1, to provide the necessary electrical power for the data transmitter 30, data receiver 40, data processor 50, and data display. Such a power source could be rechargeable batteries, or replaceable batteries 110, which may be incorporated into the housing 20, and are electrically connected to each device or system element requiring power.

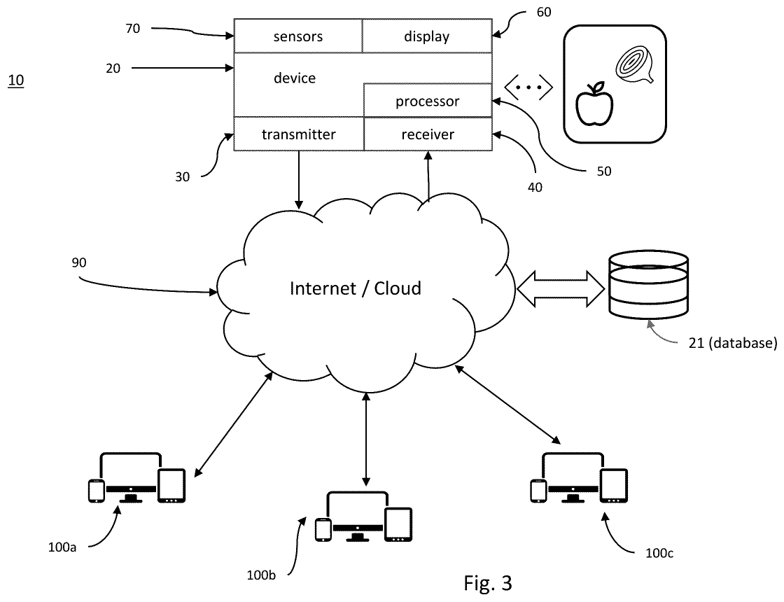

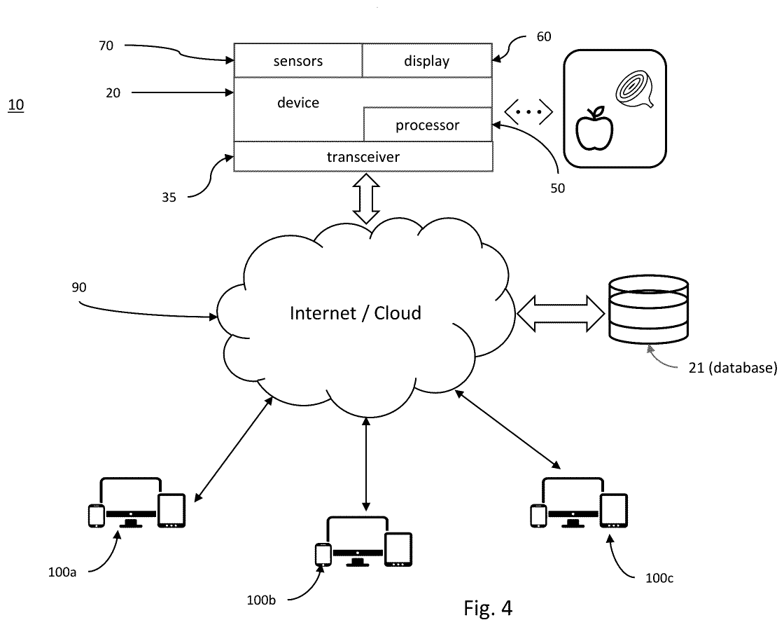

Another embodiment of the monitoring and reporting device 10, providing further data analytics, and illustrated in FIG. 3, again comprises the housing 20 with a data transmitter 30, a data receiver 40, a data processor 50, a data display 60, and further incorporates at least one sensor 70 to measure one or more metrics that are relevant to the products being monitored. The metrics monitored by the device 10 would be those metrics most relevant to the showing or providing of data associated with the condition and state of the products. By way of non-exclusive example, such metrics may include direct metrics of the products, such as quantity, weight, volume, and size. Such metrics may also include indirect metrics relating to the products, such as interior or surrounding temperature, humidity, pressure and gases within the housing. In a further embodiment of the monitoring and reporting device 10, as shown in FIG. 4, the data transmitter and receiver may be combined into a transceiver 35.

The range of sensors that may be incorporated into the monitoring device 10 are only limited by the type of metrics that are relevant to the products or goods being measured, and the availability of sensors to detect and measure those metrics. Typical sensors 70 that are relevant for monitoring and reporting on the status and condition of perishable food products include, without limitation, product quantity, weight, volume, size, and color, and housing interior conditions, such as temperature, humidity, pressure, and gases. Each of the sensors measures the respective metric and transmits a respective data signal for that metric to the processor 50 for recording or storing in memory, further analysis, and then reporting to a remote/Cloud data base or repository 21, and reporting to one or more connected user interface devices 100a, 100b, 100c, which is illustrated in FIGS. 1 through 4.

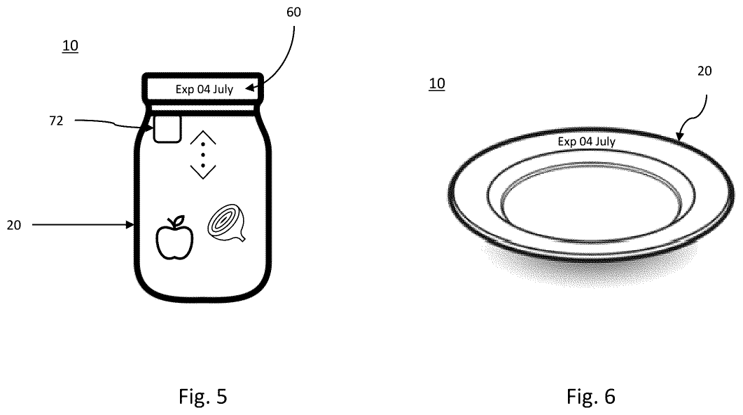

In one embodiment, an effective sensor for measuring or recording certain of the noted metrics would be one or more cameras 72. A camera 72, as shown in FIG. 5, or multiple cameras, could be used to record digital images of the product or products, and through comparative analytics, identify differences in the quantity and quality of the remaining products, including the volume or size of the products, and with a color sensing camera, identify differences in the color of the products. Such color differences could be used to identify oxidation of the products, wilting of organic products, or merely discoloration due to food products becoming overly ripe.

A camera 72 could also provide a means of recording a QR code, or bar code, or be used to read an expiration or “use-by” date that may be associated with, or attached to the product or the product packaging. Similarly, a camera or multiple cameras 72 could record visual images of any products or items within the device. Such images could be recorded, transmitted, or stored for later usage and analytics, including through use of such images to create 3-D virtual renderings or representations of the products within the device.

In other embodiments, the monitoring device 10 could include sensors that detect when the housing or container is accessed, opened, closed, or simply moved. Such sensors could be simple open and close switches that actuate when the housing opened or closed, or could be motion sensors that are actuated upon sensing motion of the housing 20. Sensing and recording such an “access” metric would allow monitoring of how often the housing or container was opened, which would provide data as to the frequency of accessing the perishable product. With such metric data, showing the frequency of accessing a container, and with an associated algorithm, the system could provide users with trending data, and prediction of time before the product is completely used up.

Similar to the monitoring of product usage, by monitoring product weight, volume, and simply by monitoring expiration date, the system 10 could also provide data useful for inventory monitoring and control. For example, by monitoring product weight, when the weight being measured is less than, or approaching a set threshold, a data signal or message may be generated by the processor 50, and transmitted to the user, through the user's display devices 100a, that the product is in need of being replenished.

In another aspect of such inventory control element and embodiment, the system 10 could monitor product usage, and provide automatic instructions for re-ordering or repurchasing of the product that has been used up, or has expired. Further, as part of inventory control and management, with proper identification of local or macro locations, such as through geo-locating elements, the inventory control processing could provide information about locations of product, and whether one or more locations, as compared to other locations are running low of, or do not have sufficient product for the user's needs. One application of such an inventory control and management process could be within a supermarket location, and could provide for communication between the monitoring system 10 and a “smart shopping cart” or other data retrieving system that records movement and location of the monitoring system.

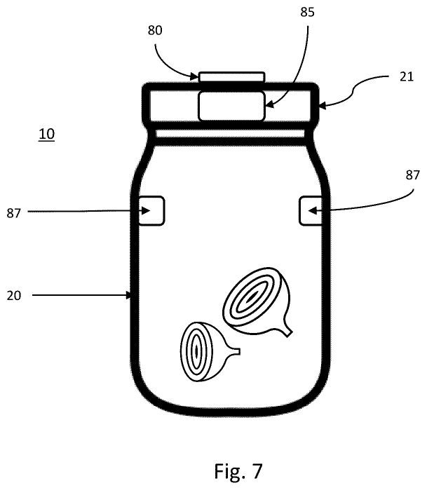

Similarly, inventory control may be further provided through use of the monitoring device 10 that further incorporates one or more radio frequency identification (“RFID”) tags 80. Such RFID tags 80 may be passive or active, where such active tags would need to further have a power source, such as a battery. The purpose and usage of the RFID tags 80 would be for users to track the location and movement of the monitoring device 10, and thereby monitor the location and movement of any products or goods that are being maintained within the monitoring device 10. The RFID tag 80 could be attached to the monitoring device, as illustrated in FIG. 7.

A further embodiment of the monitoring device 10 includes as part of the housing 20, a locking cap or top 21, as illustrated in FIG. 7. For configurations where the product being monitored is to be secured, the locking cap 21 may be remotely controlled through a user's device 100a. In such a configuration, a security code may be provided only to certain users, who are allowed access to the monitoring device 10, and accordingly limit and monitor access to the perishable product within the device 10. A locking cap 21 used with the monitoring device may have particular application for the storage of pharmaceuticals, and the desire to prevent children from having ready access to such products. In alternative similar embodiments, the locking cap or top 21 may be configured to be bottom of the housing 20, or a side access point.

In different embodiments, the monitoring and reporting device 10 may be configured into a variety of housings or devices, and in a wide selection of shapes and sizes. In one embodiment of the monitoring device 10, the housing 20 could simply be a container, such as a jar or a plastic resealable container, as shown in FIGS. 5, 7, and 8, with a data display 60. Alternatively, as shown in FIG. 6, the housing 20 may be a plate or scale upon which the perishable product being monitored is placed. When configured or incorporated into a sealable container, the housing 20 elements could be configured as part of the container top or cap, or may be incorporated into the container body section. Where the monitoring device 10 is configured as a plate or scale, the housing 20 elements would be incorporated into the plate body.

In a further embodiment of the monitoring device, the housing 20 could be configured or manufactured as a sealable plastic or flexible material bag that has the data transmitter 30, data receiver 40, data processor 50, data display 60, and a plurality of sensors 70 attached to the interior or exterior of the plastic bag.

As an additional feature to the monitoring device 10, where the housing is configured as a sealable container, the housing may further include an air evacuation device 85, as illustrated in FIG. 7, that is capable of removing air from within the sealed container. By removing the interior air, and primarily the oxygen from within the container, any degrading or deterioration of the perishable product is likely to be reduced, thereby extending the “life” of perishable product. Obviously, for this embodiment where the interior air may be removed, the container must be manufactured with sufficient strength to prevent collapse or breakage of the container due to exterior pressure. Many such containers exist including by way of example, the vacuum system by Zwilling™.

In a further embodiment of the monitoring and reporting device 10, bacterial control devices 87 may also be incorporated into the device housing 20, as illustrated in FIG. 7. For example, bacterial control lighting 87 may be part of the housing 20, and may be actuated at preset intervals, or may be remotely controlled by a user through his or her remote handheld device 100a. Such lighting could include ultraviolet lighting, such as UV-C wavelength light (200 to 280 nanometers), or possibly longer wavelength UV-B or UV-A light (280 to 400 nm). The anti-bacterial and germicidal protections available from UV light are well-known in the art. Such lighting systems may be readily incorporated into the housing 20, with the lighting configured to be directed at the perishable product being monitored and contained.

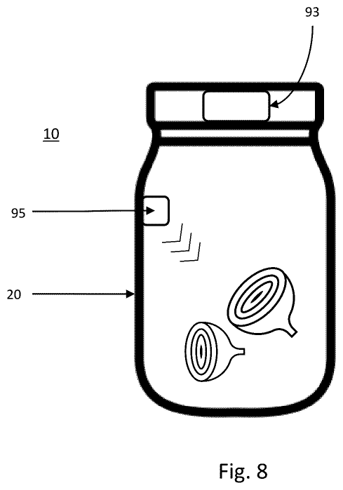

Another enhanced embodiment of the monitoring and reporting device and system 10, as illustrated in FIG. 8, may include a motion sensor 93 incorporated into the device housing 20, along with a lighting element 95, such that when the motion sensor 93 senses motion, for example, when the housing is opened, then a signal is transmitted to provide power to the lighting element 95 to illuminate the area or volume of the device 10 where the product being monitored is stored.

While preferred embodiments of the inventive devices and systems for monitoring and reporting the status of perishable products have been described and disclosed, in particular with reference to certain figures and exemplary embodiments of the monitoring and reporting devices and systems, such exemplary representations are not to be construed as limiting the scope of application of the inventive devices and systems. By way of example, other types of housing may be used as the monitoring device, other than a cap or a plate, including by way of example, a sealable container, or a reusable baggie. Moreover, other sensors or sensor types, other than those disclosed, including temperature, humidity, weight, and visual, may be used to provide additional metrics relating to the items being monitored and reported upon, and may be incorporated into an embodiment of the monitoring and reporting device and system. Further, other mechanisms may be used for entering data into, or controlling the data processor 50, including voice activated devices.

Further, while described and disclosed primarily for use with the monitoring and reporting of perishable food items, the inventive monitoring and reporting devices and systems have equally effective application to monitor and report on other perishable items, or items that have a “shelf-life,” including, by way of example, pet food products, pharmaceuticals, adhesives, acids, and other consumable and non-consumable chemical compositions.

It will be recognized by those skilled in the art that other modifications, substitutions, and/or other applications are possible, and all such modifications, substitutions and applications are within the true scope and spirit of the present invention. It is likewise understood that the above disclosure and attached claims are intended to cover all such modifications, substitutions, and/or applications.

What is claimed is:

1. A device for reporting status of perishable items, said device comprising:
a housing with each of the following incorporated into said housing;
i. a data communications receiver;
ii. a data communication transmitter; and
iii. a data processor operating machine-readable media in data communication with said data communications receiver, and said data communications transmitter; and
iv. a power source for said data communications receiver, data communications transmitter, and said data processor;
wherein said device is in data communication with at least one remote user display device.

2. The device for reporting status of perishable items, as described in claim 1, wherein said housing is a container top.

3. The device for reporting status of perishable items, as described in claim 1, further comprising a data display incorporated into said housing.

4. The device for reporting status of perishable items, as described in claim 1, wherein said data communications receiver and said data communications transmitter are comprised within a unitary transceiver.

5. The device for reporting status of perishable items, as described in claim 1, wherein said data processor operating said machine-readable media controls a countdown timer displayed by said data display.

6. The device for reporting status of perishable items, as described in claim 1, further comprising a data input device for manually entering data associated with said perishable items.

7. The device for reporting status of perishable items, as described in claim 1, further comprising a data interface device for entering metric data associated with said perishable items to said data processor.

8. The device for reporting status of perishable items, as described in claim 7, wherein said metric data associated with said perishable items comprises at least one of inventory, weight, volume, duration within device, number of times said device is accessed.

9. The device for reporting status of perishable items, as described in claim 1, further comprising at least one light emitter to control bacterial growth on said perishable items.

10. The device for reporting status of perishable items, as described in claim 9, wherein said at least one light emitter is an ultraviolet light emitter.

11. A device to monitor, analyze, record, and report condition and status of perishable food items, said device, comprising:
a housing;
a data input device for manually entering metric data associated with said food items into said device;
a data display device;
a data communications transceiver;
a processor operating machine-readable media executing at least one algorithm for analyzing metrics associated with said food items;
a power source for said data display, data communications transceiver, and said data processor; and
wherein said device is in data communication with at least one remote user display device.

12. The device to monitor, analyze, and report condition and status of perishable food items, as described in claim 11, wherein said housing is a container or container top.

13. The device to monitor, analyze, and report condition and status of perishable food items, as described in claim 11, wherein said data processor operating said machine-readable media controls a countdown timer displayed by said data display.

14. The device to monitor, analyze, and report condition and status of perishable food items, as described in claim 11, wherein said metric data associated with said food items comprises at least one of inventory, weight, volume, duration within device, number of times said device is accessed.

15. A system to monitor, analyze, and report condition and status of perishable food items, comprising:
An apparatus housing a plurality of sensors located within sealable opening associated with said apparatus, to sense and monitor metrics associated with said condition and status of said perishable food items, wherein said plurality of sensors sense at least one of a group of metrics associated with said perishable food items, said at least one of a group of metrics comprising expiration date, weight, volume, color of the perishable food items, and temperature, humidity, and emitted gases within an internal volume of said apparatus (methane, oxygen, carbon dioxide),
A microprocessor in data communication with each of said plurality of sensors, wherein said microprocessor records and analyzes a condition and status of said perishable food items based upon at least one of said sensed metrics;
A transmitter in data communication with said microprocessor;
A power source for said plurality of sensors, microprocessor and transmitter; and
At least one remote user device and receiver in data communication with said transmitter, and wherein said transmitter transmits said condition and status of said perishable food items to said at least one remote user device and receiver.

16. The system to monitor, analyze, and report condition and status of perishable food items device to monitor and report condition and status of perishable food items, as described in claim 15, further comprising at least one light emitter to control bacterial growth on said perishable food items.

17. The system to monitor, analyze, and report condition and status of perishable food items device to monitor and report condition and status of perishable food items, as described in claim 15, wherein said apparatus is an enclosure having a sealable opening, and said enclosure defining an internal volume.

18. The system to monitor, analyze, and report condition and status of perishable food items device to monitor and report condition and status of perishable food items, as described in claim 15, wherein said apparatus further comprises a means for evacuating air from within said defined internal volume.

19. The system to monitor, analyze, and report condition and status of perishable food items device to monitor and report condition and status of perishable food items, as described in claim 15, wherein said apparatus is a plate upon which said perishable food items are placed.

20. The system to monitor and report condition and status of perishable food items, as described in claim 15, wherein at least one of said plurality of sensors is a camera, bar code reader, or scanner to read at least an expiration date associated with said perishable food items.

21. A method to monitor, analyze, and report conditions and status of perishable food items, where said perishable food items are housed within a device, said housing comprising a plurality of sensors to sense at least one of a group of metrics associated with said perishable food items, a microprocessor, and a transmitter in data communication with said microprocessor, and at least one remote user interface device, said method comprising the steps of:
a. Sensing at least one of a group data metrics associated with said perishable food items;
b. Recording said sensed data metrics within memory associated with said microprocessor;
c. Analyzing said sensed data metrics to determine a condition and status of said perishable food items based upon said sensed data metrics; and
d. Reporting said analyzed data metrics to said at least one remote user interface device, wherein said analyzed data metrics include at least one of thresholds, warnings, and trends.
